(19) United States
(12) Patent Application Publication
(10) Pub. No.: US 2003/0225366 A1
(43) Pub. Date: Dec. 4, 2003

(54) Liquid venting surgical cassette

(76) Inventors: Michael D. Morgan, Costa Mesa, CA (US); Gary P. Sorensen, Irvine, CA (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/430,974

(22) Filed: May 7, 2003

Related U.S. Application Data
(63) Continuation-in-part of application No. 10/407,388, filed on Apr. 4, 2003.
Application No. 10/407,388 is a continuation-in-part of application No. 09/846,724, filed on May 1, 2001, now Pat. No. 6,572,349.
Application No. 09/846,724 is a continuation of application No. 09/437,392, filed on Nov. 10, 1999, now Pat. No. 6,293,926.
Application No. 10/407,388 is a continuation-in-part of application No. 09/925,989, filed on Aug. 9, 2001.
Application No. 09/925,989 is a continuation-in-part of application No. 09/771,945, filed on Jan. 29, 2001, now Pat. No. 6,632,214.
Application No. 09/771,945 is a division of application No. 09/387,357, filed on Aug. 31, 1999, now Pat. No. 6,261,283.

(51) Int. Cl. A61M 3/00

(52) U.S. Cl. 604/45

(57) ABSTRACT

A surgical system having a cassette with an aspirant collection chamber and an aspiration vent line that draws fluid from the aspirant collection chamber. The pressure within the collection chamber is maintained near ambient so that when the aspiration vent line is open, fluid flows from the collection chamber and into the aspiration line. Such a system does not require a second source of irrigation fluid, minimizes pressure surges into the irrigation fluid line and does not affect the fluidic performance of the aspiration system. In addition, various vent pressures can be achieved by varying the vertical position of the reservoir relative to the aspiration line.

This application is a continuation-in-part of U.S. patent application Ser. No. 10/407,388, filed Apr. 4, 2003, which is a continuation-in-part application of U.S. patent application Ser. No. 09/846,724, filed May 1, 2001, which is a continuation of U.S. patent application Ser. No. 09/437,392, filed Nov. 10, 1999, now U.S. Pat. No. 6,293,926 B1, and a continuation-in-part of U.S. patent application Ser. No. 10/153,371, filed May 28, 2002, and is a continuation-in-part application of U.S. patent application Ser. No. 09/925,989, filed Aug. 9, 2001, which is a continuation-in-part of U.S. patent application Ser. No. 09/771,945, filed Jan. 29, 2001, which is a divisional of U.S. patent application Ser. No. 09/387,357, filed Aug. 31, 1999, now U.S. Pat. No. 6,261,283 B1.

### BACKGROUND OF THE INVENTION

This invention relates generally to the field of cataract surgery and more particularly to an aspiration system for a handpiece for practicing the phacoemulsification technique of cataract removal.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

A typical ultrasonic surgical device suitable for ophthalmic procedures consists of an ultrasonically driven handpiece, an attached cutting tip, and irrigating sleeve and an electronic control console. The handpiece assembly is attached to the control console by an electric cable and flexible tubings. Through the electric cable, the console varies the power level transmitted by the handpiece to the attached cutting tip and the flexible tubings supply irrigation fluid to and draw aspiration fluid from the eye through the handpiece assembly.

The operative part of the handpiece is a centrally located, hollow resonating bar or horn directly attached to a set of piezoelectric crystals. The crystals supply the required ultrasonic vibration needed to drive both the horn and the attached cutting tip during phacoemulsification and are controlled by the console. The crystal/horn assembly is suspended within the hollow body or shell of the handpiece by flexible mountings. The handpiece body terminates in a reduced diameter portion or nosecone at the body's distal end. The nosecone is externally threaded to accept the irrigation sleeve. Likewise, the horn bore is internally threaded at its distal end to receive the external threads of the cutting tip. The irrigation sleeve also has an internally threaded bore that is screwed onto the external threads of the nosecone. The cutting tip is adjusted so that the tip projects only a predetermined amount past the open end of the irrigating sleeve. Ultrasonic handpieces and cutting tips are more fully described in U.S. Pat. Nos. 3,589,363; 4,223,676; 4,246,902; 4,493,694; 4,515,583; 4,589,415; 4,609,368; 4,869,715; 4,922,902; 4,989,583; 5,154,694 and 5,359,996, the entire contents of which are incorporated herein by reference.

In use, the ends of the cutting tip and irrigating sleeve are inserted into a small incision of predetermined width in the cornea, sclera, or other location. The cutting tip is ultrasonically vibrated along its longitudinal axis within the irrigating sleeve by the crystal-driven ultrasonic horn, thereby emulsifying the selected tissue in situ. The hollow bore of the cutting tip communicates with the bore in the horn that in turn communicates with the aspiration line from the handpiece to the console. A reduced pressure or vacuum source in the console draws or aspirates the emulsified tissue from the eye through the open end of the cutting tip, the cutting tip and horn bores and the aspiration line and into a collection device. The aspiration of emulsified tissue is aided by a saline flushing solution or irrigant that is injected into the surgical site through the small annular gap between the inside surface of the irrigating sleeve and the cutting tip.

During surgery, the hollow, resonating tip can become occluded. During occlusion, vacuum can build in the aspiration line downstream of the occlusion. When the occlusion eventually breaks apart, this pent up vacuum is released into the eye which can, depending upon the amount of vacuum, draw a significant amount of fluid from the eye, thereby increasing the risk of anterior chamber collapse. To address this concern, modern surgical console can detect increases in aspiration line vacuum beyond normal operating parameters and therefore predict occlusions. These consoles can then either stop or slow the aspiration pump, or sound an alarm so that the surgeon can take appropriate precautions.

The cassettes used in modern consoles also allow the aspiration line to be vented, either to atmosphere or to a liquid so as to reduce or eliminate vacuum surge upon occlusion break. Prior art air vented cassettes allow ambient air to enter the aspiration line, however, venting air into the aspiration line changes the fluidic performance of the aspiration system. Liquid venting systems allow irrigation fluid to bleed into the aspiration line, thereby reducing any impact on the fluidic performance of the aspiration system. Liquid venting cassettes are more fully described in U.S. Pat. Nos. 4,832,685 and 4,935,005 (Haines) and U.S. Pat. No. 4,713,051 (Steppe, et al.), the entire contents of which being incorporated herein by reference. When higher aspiration vacuums are used, cassettes that vent the aspiration line to the irrigation line can cause high pressure surges in the irrigation line. Other systems provide a separate source of irrigation fluid to vent the aspiration line, requiring the use of two irrigation fluid sources and increasing the cost and complexity of the system.

Therefore, a need continues to exist for a simple surgical system that allows rapid venting of excess aspiration vacuum without introducing pressure variations in the irrigation line or the downstream aspiration line.

### BRIEF SUMMARY OF THE INVENTION

The present invention improves upon the prior art by providing a surgical system having a cassette with an aspirant collection chamber and an aspiration vent line that draws fluid from the aspirant collection chamber. The pressure within the collection chamber is maintained near ambient so that when the aspiration vent line is open, fluid flows from the collection chamber and into the aspiration line. Such a system does not require a second source of irrigation fluid, minimizes pressure surges into the irrigation fluid line and does not affect the fluidic performance of the aspiration system. In addition, various vent pressures can be achieved by varying the vertical position of the reservoir relative to the aspiration line.

Accordingly, one objective of the present invention is to provide a surgical system having a aspiration line vent.

Another objective of the present invention is to provide a surgical system having a cassette that allows the aspiration line to be vented of excess vacuum.

Another objective of the present invention is to provide a surgical system having a cassette that vents the aspiration line to an aspirant collection chamber.

Another objective of the present invention is to provide a surgical system that vents the aspiration line without introducing pressure surges in the irrigation line.

Another objective of the present invention is to provide a surgical system that vents the aspiration line without affecting the fluidic performance of the aspiration system.

These and other advantages and objectives of the present invention will become apparent from the detailed description and claims that follow.

### BRIEF DESCRIPTION OF THE DRAWING

FIG. 1 is a schematic illustration of a first embodiment of the system and cassette of the present invention.

FIG. 2 is an enlarged, partially cut away view of a cassette that may be used with the system of the present invention.

FIG. 3 is a schematic illustration of a second embodiment of the system and cassette of the present invention.

FIG. 4 is an enlarged perspective view of the second embodiment of a cassette that may be used with the present invention.

FIG. 5 is an enlarged perspective view of a cassette that may be used with the present invention.

### DETAILED DESCRIPTION OF THE INVENTION

System 10 of the present invention generally includes surgical console 12 and fluid management system (FMS) or cassette 14. Console 12 may be any suitably modified commercially available surgical console, such as the SERIES TWENTY THOUSAND® LEGACY® or ACCURUS® surgical systems available from Alcon Laboratories, Fort Worth, Tex. Cassette 14 may be any suitably modified commercially available surgical cassettes, such as those described in U.S. Pat. Nos. 5,267,956, 5,364,342 and 5,499,969 (Beuchat, et al.) and U.S. Pat. No. 5,899,674 (Jung, et al.), the entire contents of which being incorporated herein by reference. Cassette 14 is held in operative association with console 12 by means well-known in art.

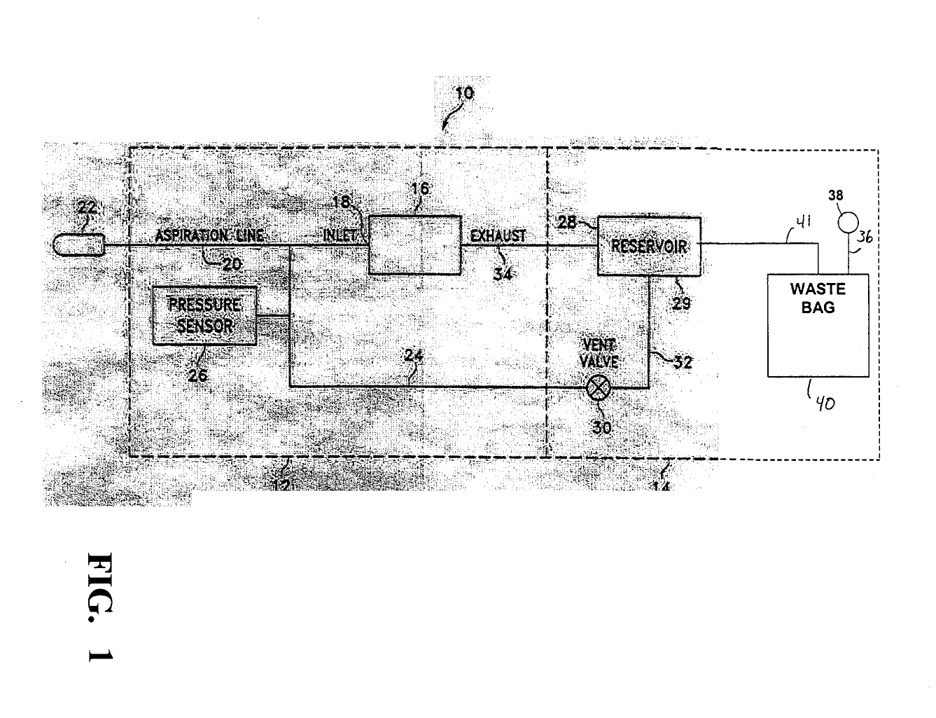

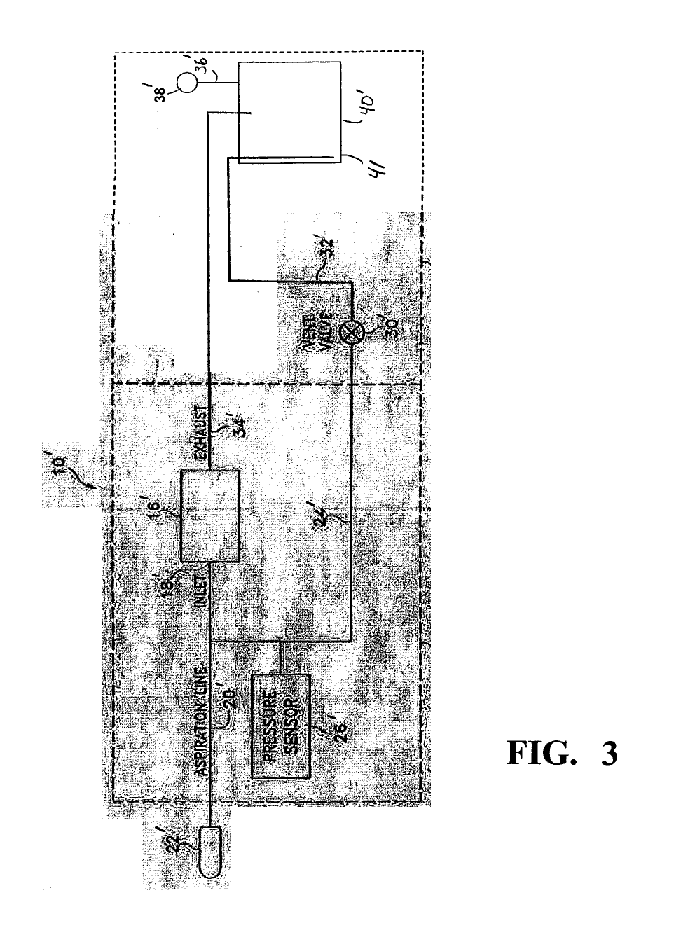

As seen in FIGS. 1 and 3, console 12 or 12′ generally contains aspiration pump mechanism 16 or 16′, which may be any suitable flow or vacuum based pump, such pumps being widely known in the art. For example, pump mechanism 16 or 16′ may be a peristaltic pump roller head that interacts with a peristaltic pump tube formed by aspiration line 20 or 20′ and aspiration exhaust line 34 or 34′. Aspiration line 20 or 20′ is connected to surgical handpiece 22 or 22′ on one end and end 18 or 18′ of aspiration line 20 or 20′ opposite handpiece 22 or 22′ interacts with pump mechanism 16 or 16′ so as to draw fluid through handpiece 22 or 22′. Aspiration line 20 or 20′ is intersected between handpiece 22 or 22′ and inlet 18 or 18′ by aspiration vent line 24 or 24′. In fluid communication with aspiration vent line 24 or 24′ is pressure sensor 26 or 26′, which may be one of a variety of invasive or non-invasive pressure sensors well-known in the art.

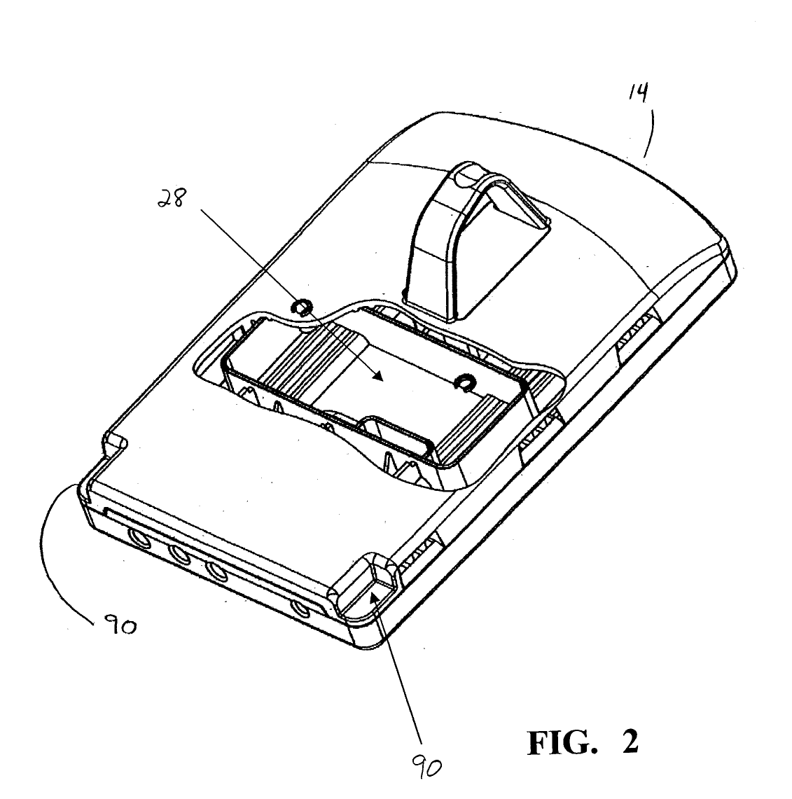

Cassette 14 generally contains fluid reservoir 28. As best seen in FIG. 2, reservoir 28 may be integrally molded within cassette 14. Extending from reservoir 28 at or near bottom 29 is aspiration vent line 32, which fluidly connects to aspiration vent line 24 through vent valve 30. Aspirant or exhaust from pump mechanism 16 is directed into reservoir 28 through aspiration exhaust line 34. Reservoir 28 may also vent to ambient through fluid line 41, waste bag 40 and vent line 36, which may contain antimicrobial filter 38.

As discussed above, while it is preferred that pump mechanism 16 be a peristaltic roller head and aspiration line 20 and aspiration exhaust line 34 be formed in one continuous length so as to form a peristaltic pump tube that interacts with pump mechanism 16, one skilled in the art will recognize that aspiration line 20 and aspiration exhaust line may be formed as a separate piece or pieces or may be formed integrally with cassette 14 and that pump mechanisms 16 other that peristaltic pump roller heads may be used, such as linear peristaltic pumps.

In addition, pressure sensor 26 is depicted as being contained within console 12. One skilled in the art will recognize that portions of pressure sensor 26, such as a pressure diaphragm (not shown) may be contained in or on cassette 14 and interact with a force transducer or other means (not shown) contained within console 12.

In use, cassette 14 is installed on or within console 12 and held in operative association with console 12 by means well-known in the art. System 10 is primed initially with clean surgical fluid so that a small amount of fluid fills reservoir 28. During surgery, pump mechanism 16 draws aspirant through handpiece 22 and into reservoir 28. If the vacuum within aspiration line 20 is too high and needs to be vented, vent valve 30 is opened allowing aspirant to be drawn off of bottom 29 of reservoir 28 (reservoir 28 being at or near ambient) and into aspiration line 20 (which contains a vacuum) through aspiration vent line 24. One skilled in the art will recognize that by varying the vertical position of reservoir 28 relative to aspiration line 20, various vent head pressures may be achieved. Reservoir 28 may be relatively small, and not capable of holding all of the aspirant collected during surgery. Waste bag 40 may be used to drain and hold excess fluid from reservoir 28, through fluid line 41 above bottom 29 of reservoir 28. Waste bag 40 may be integrally formed within cassette 14 or may be formed as a separate collapsible bag attached to cassette 14, as is well-known in the art.

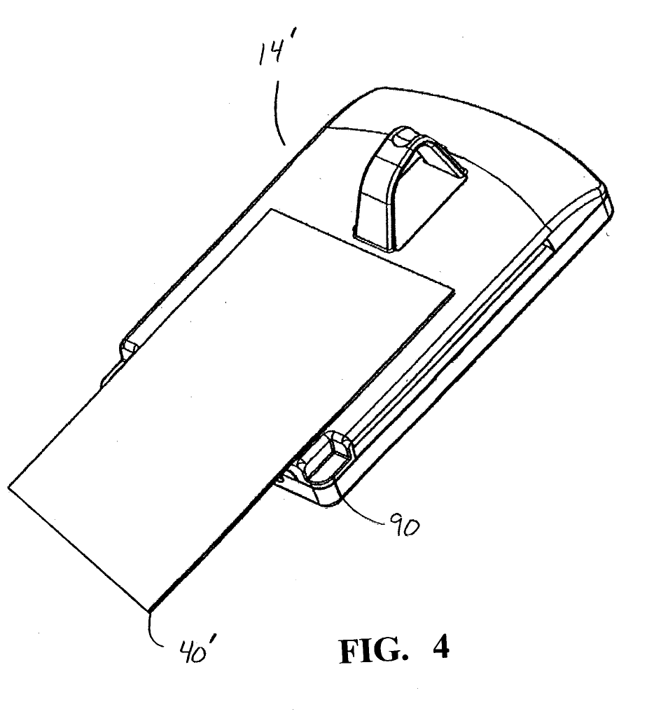

As best seen in FIGS. 3 and 4, in a second embodiment of the system 10′ of the present invention, cassette 14′ does not contain an integral fluid reservoir, but aspiration vent line 32′ extends into drain bag 40′ so as to draw fluid off of bottom 41 of drain bag 40′. Vent line 32′ fluidly connects to aspiration vent line 24′ through vent valve 30′. Aspirant or exhaust from pump mechanism 16′ is directed into drain bag 40′ through aspiration exhaust line 34′. Preferably, in this embodiment, drain bag 40′ is as a separate collapsible bag attached to cassette 14′, as is well-known in the art. Drain bag 40′ may also vent to ambient through vent line 36′, which may contain antimicrobial filter 38′.

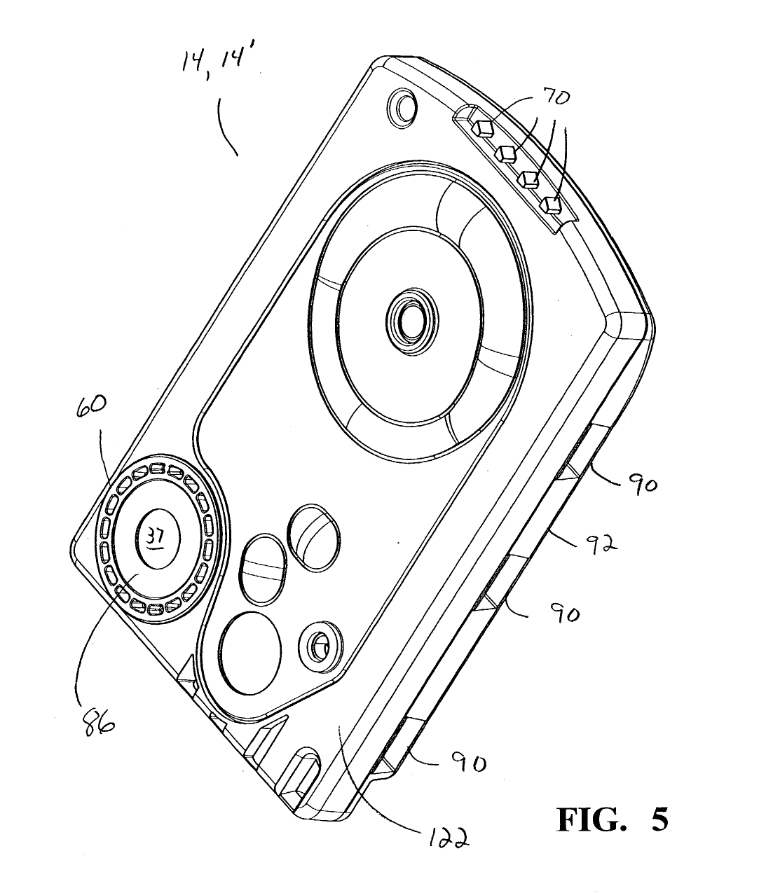

As best seen in FIG. 5, cassette 14 or 14′ of the present invention contains aspiration pressure sensor interface 86. Interface 86 may consist of thin diaphragm 37 preferably made of metal or rigid plastic. An external loadcell (not shown) located on console 12 or 12″ is used to deflect diaphragm 37 to a predetermined initial force load. Changes in pressure on the internal side of diaphragm 37 opposite the loadcell will cause a change in force on the loadcell that can be correlated to the pressure level on interface 86. Forces on cassette 14 or 14′ during use can cause flexing of substrate 122. Such flexing can cause inaccurate reading from the load cell used in conjunction with pressure sensor interface 86. In order to minimize such inaccuracies, substrate 122 contains a raised boss 60 around pressure sensor interface 86. When substrate 122 is clamped in place against a rigid mounting member (not shown) on console 12 or 12″, boss 60 helps to stabilize the area around pressure sensor interface 86 and isolate pressure sensor interface 86 from forces acting on other regions of substrate 122. In addition, cassette 14 or 14′ may contain one or more plurality of latching surfaces 90 per side that cooperate with a latching mechanism, such as the latching mechanism described in commonly-owned U.S. patent application Ser. No. 10/132,797, filed Apr. 25, 2002, to assist in holding cassette 14 or 14′ firmly within console 12 or 12″ further reducing the possibility of flexing of cassette 14 or 14′. Latching surfaces 90 preferably are located along sides 92 of substrate 122 and preferably, are formed as indentations within sides 92 of substrate 122. Such a construction helps to prevent the possibility of the latching mechanism capturing the finger of a user.

Cassette 14 or 14′ may also contain a plurality of identifying tabs 70 projecting from housing substrate 122. Tabs 70 may be generally of the shape described in U.S. Pat. No. 6,059,544 (Jung, et al.) and may be of variable opaqueness, from completely opaque to partially translucent to relatively clear. The opaqueness of tabs 70 may be used by console 12 or 12″ to identify the particular cassette 14 or 14′ being used from a variety of suitable cassette 14 or 14′ in a manner similar to that disclosed in U.S. Pat. No. 6,059,544 (Jung, et al.), the entire contents of which being incorporated herein by reference.

This description is given for purposes of illustration and explanation. It will be apparent to those skilled in the relevant art that changes and modifications may be made to the invention described above without departing from its scope or spirit.

We claim:

1. A cassette, comprising:
a) a body having an exterior and a fluid reservoir;
b) an aspiration vent line extending through the body from the reservoir at or near a bottom of the reservoir;
c) an aspiration pressure sensor interface on the exterior of the body; and
d) a raised boss around the pressure sensor interface.

2. The cassette of claim 1 wherein the reservoir is located on the exterior of the body.

3. The cassette of claim 1 wherein the body has an interior and the fluid reservoir is located in the interior of the body.

4. The cassette of claim 1 wherein the cassette further comprises a plurality of identifying tabs projecting from the exterior of the body.

5. A cassette, comprising:
a) a body having an exterior and an interior;
b) a fluid reservoir located in the interior of the body;
c) an aspiration vent line extending through the body from the reservoir at or near a bottom of the reservoir;
d) an aspiration pressure sensor interface on the exterior of the body;
e) a raised boss around the pressure sensor interface; and
f) a plurality of identifying tabs projecting from the exterior of the body.

6. A cassette, comprising:
a) a body having a fluid reservoir and an exterior, the exterior having sides;
b) an aspiration vent line extending through the body from the reservoir at or near a bottom of the reservoir;
c) an aspiration pressure sensor interface on the exterior of the body; and
d) a plurality of latching surfaces formed in the sides of the body.

7. The cassette of claim 6 wherein the reservoir is located on the exterior of the body.

8. The cassette of claim 6 wherein the body has an interior and the fluid reservoir is located in the interior of the body.

9. The cassette of claim 6 wherein the cassette further comprises a plurality of identifying tabs projecting from the exterior of the body.

10. A cassette, comprising:
a) a body having a reservoir and an exterior, the exterior having sides;
b) an aspiration vent line extending through the body from the reservoir at or near a bottom of the reservoir;
c) an aspiration pressure sensor interface on the exterior of the body;
d) a raised boss around the pressure sensor interface;
e) a plurality of identifying tabs projecting from the exterior of the body; and
f) a plurality of latching surfaces formed in the sides of the body.

11. The cassette of claim 10 wherein the reservoir is located on the exterior of the body.

12. The cassette of claim 10 wherein the body has an interior and the fluid reservoir is located in the interior of the body.
